(19) United States
(12) Patent Application Publication
(10) Pub. No.: US 2009/0118377 A1
(43) Pub. Date: May 7, 2009

(54) APOGOSSYPOLONE AND THE USES THEREOF

(75) Inventors: Shaomeng Wang, Saline, MI (US); Jianyong Chen, Ann Arbor, MI (US); Zaneta Nikolovska-Coleska, Ann Arbor, MI (US); Dajun Yang, Rockville, MD (US)

Correspondence Address: Casimir Jones, S.C., 440 Science Drive, Suite 203, Madison, WI, 53711, US

(73) Assignee: The Regents of the University of Michigan, Ann Arbor, MI (US)

(21) Appl. No.: 12/352,399

(22) Filed: Jan. 12, 2009

Related U.S. Application Data
(62) Division of application No. 11/265,576, filed on Nov. 2, 2005.
(60) Provisional application No. 60/624,437, filed on Nov. 2, 2004.

(51) Int. Cl. A61K 31/122; C12N 5/00; A61P 35/00

(52) U.S. Cl. 514/681; 435/375

(57) ABSTRACT

The invention relates to the compound apogossypolone and salts and prodrugs thereof. Apogossypolone functions as an inhibitor of Bcl-2 family proteins. The invention also relates to the use of apogossypolone for inhibiting hyperproliferative cell growth, for inducing apoptosis in cells and for sensitizing cells to the induction of apoptotic cell death.

This application claims priority to U.S. Provisional Application Ser. No. 60/624,437, filed Nov. 2, 2004, herein incorporated by reference in its entirety.

### BACKGROUND OF THE INVENTION

1. Field of the Invention

This invention is in the field of medicinal chemistry. In particular, the invention relates to the compound apogossypolone and salts and prodrugs thereof. Apogossypolone functions as an inhibitor of Bcl-2 family proteins. The invention also relates to the use of apogossypolone for inhibiting hyperproliferative cell growth, for inducing apoptosis in cells and for sensitizing cells to the induction of apoptotic cell death.

2. Related Art

The aggressive cancer cell phenotype is the result of a variety of genetic and epigenetic alterations leading to deregulation of intracellular signaling pathways (Ponder, Nature 411:336 (2001)). The commonality for all cancer cells, however, is their failure to execute an apoptotic program, and lack of appropriate apoptosis due to defects in the normal apoptosis machinery is a hallmark of cancer (Lowe et al., Carcinogenesis 21:485 (2000)). Most of the current cancer therapies, including chemotherapeutic agents, radiation, and immunotherapy, work by indirectly inducing apoptosis in cancer cells. The inability of cancer cells to execute an apoptotic program due to defects in the normal apoptotic machinery is thus often associated with an increase in resistance to chemotherapy, radiation, or immunotherapy-induced apoptosis. Primary or acquired resistance of human cancer of different origins to current treatment protocols due to apoptosis defects is a major problem in current cancer therapy (Lowe et al., Carcinogenesis 21:485 (2000); Nicholson, Nature 407:810 (2000)). Accordingly, current and future efforts towards designing and developing new molecular target-specific anticancer therapies to improve survival and quality of life of cancer patients must include strategies that specifically target cancer cell resistance to apoptosis. In this regard, targeting crucial negative regulators that play a central role in directly inhibiting apoptosis in cancer cells represents a highly promising therapeutic strategy for new anticancer drug design.

Two classes of central negative regulators of apoptosis have been identified. The first class of regulators is the inhibitor of apoptosis proteins (IAPs) (Deveraux et al., Genes Dev. 13:239 (1999); Salvesen et al., Nat. Rev. Mol. Cell. Biol. 3:401 (2002)). IAP proteins potently suppress apoptosis induced by a large variety of apoptotic stimuli, including chemotherapeutic agents, radiation, and immunotherapy in cancer cells.

The second class of central negative regulators of apoptosis is the Bcl-2 family of proteins (Adams et al., Science 281:1322 (1998); Reed, Adv. Pharmacol. 41:501 (1997); Reed et al., J. Cell. Biochem. 60:23 (1996)). Bcl-2 is the founding member of the family and was first isolated as the product of an oncogene. The Bcl-2 family now includes both anti-apoptotic molecules such as Bcl-2 and Bcl-xL and pro-apoptotic molecules such as Bax, Bak, Bid, and Bad. Bcl-2 and Bcl-xL are overexpressed in many types of human cancer (e.g., breast, prostate, colorectal, lung), including Non-Hodgkin's lymphoma, which is caused by a chromosomal translocation (t14,18) that leads to overexpression of Bcl-2. This suggests that many cancer cell types depend on the elevated levels of Bcl-2 and/or Bcl-xL to survive the other cellular derangements that simultaneously both define them as cancerous or pre-cancerous cells and cause them to attempt to execute the apoptosis pathway. Also, increased expression of Bcl-2 family proteins has been recognized as a basis for the development of resistance to cancer therapeutic drugs and radiation that act in various ways to induce cell death in tumor cells.

Bcl-2 and Bcl-xL are thought to play a role in tumor cell migration and invasion, and therefore, metastasis (Amberger et al., Cancer Res. 58:149 (1998); Wick et al., FEBS Lett, 440:419 (1998); Mohanam et al., Cancer Res. 53:4143 (1993); Pedersen et al., Cancer Res., 53:5158 (1993)). Bcl-2 family proteins appear to provide tumor cells with a mechanism for surviving in new and non-permissive environments (e.g., metastatic sites), and contribute to the organospecific pattern of clinical metastatic cancer spread (Rubio, Lab Invest. 81:725 (2001); Fernandez et al., Cell Death Differ. 7:350 (2000)). Anti-apoptotic proteins such as Bcl-2 and/or Bcl-xL are also thought to regulate cell-cell interactions, for example through regulation of cell surface integrins (Reed, Nature 387:773 (1997); Frisch et al., Curr. Opin. Cell Biol. 9:701 (1997); Del Bufalo et al., FASEB J. 11:947 (1997)).

Therapeutic strategies for targeting Bcl-2 and Bcl-xL in cancer to restore cancer cell sensitivity and overcome resistance of cancer cells to apoptosis have been extensively reviewed (Adams et al., Science 281:1322 (1998); Reed, Adv. Pharmacol. 41:501 (1997); Reed et al., J. Cell. Biochem. 60:23 (1996)). Currently, Bcl-2 antisense therapy is in several Phase III clinical trials for the treatment of solid and non-solid tumors.

Gossypol is a naturally occurring double biphenolic compound derived from crude cotton seed oil (Gossypium sp.). Human trials of gossypol as a male contraceptive have demonstrated the safety of long term administration of these compounds (Wu, Drugs 38:333 (1989)). Gossypol has more recently been shown to have some anti-proliferative effects (Flack et al., J. Clin. Endocrinol. Metab. 76:1019 (1993); Bushunow et al., J. Neuro-Oncol. 43:79, (1999); Van Poznak et al., Breast Cancer Res. Treat. 66:239 (2001)). (−)-Gossypol and its derivatives recently have been shown to be potent inhibitors of Bcl-2 and Bcl-xL and to have strong anti-cancer activity (U.S. Patent Application No. 2003/0008924).

### SUMMARY OF THE INVENTION

It is generally accepted that the inability of cancer cells or their supporting cells to undergo apoptosis in response to genetic lesions or exposure to inducers of apoptosis (such as anticancer agents and radiation) is a major factor in the onset and progression of cancer. The induction of apoptosis in cancer cells or their supporting cells (e.g., neovascular cells in the tumor vasculature) is thought to be a universal mechanism of action for virtually all of the effective cancer therapeutic drugs or radiation therapies on the market or in practice today. One reason for the inability of a cell to undergo apoptosis is increased expression and accumulation of anti-apoptotic Bcl-2 family proteins.

The present invention contemplates that exposure of animals suffering from cancer to therapeutically effective amounts of drug(s) (e.g., small molecules) that inhibit the function(s) of anti-apoptotic Bcl-2 family proteins will kill cancer cells or supporting cells outright (those cells whose continued survival is dependent on the overactivity of one or more Bcl-2 family proteins) and/or render such cells as a population more susceptible to the cell death-inducing activity of cancer therapeutic drugs or radiation therapies. The present invention contemplates that inhibitors of anti-apoptotic Bcl-2 family proteins satisfy an unmet need for the treatment of multiple cancer types, either when administered as monotherapy to inhibit hyperproliferation (e.g., by inducing apoptosis) in cancer cells dependent on anti-apoptotic Bcl-2 family protein function, or when administered in a temporal relationship with other cell death-inducing cancer therapeutic drugs or radiation therapies so as to render a greater proportion of the cancer cells or supportive cells susceptible to executing the apoptosis program compared to the corresponding proportion of cells in an animal treated only with the cancer therapeutic drug or radiation therapy alone.

In certain embodiments of the invention, combination treatment of animals with a therapeutically effective amount of a compound of the present invention and a course of an anticancer agent or radiation produces a greater tumor response and clinical benefit in such animals compared to those treated with the compound or anticancer drugs/radiation alone. Put another way, because the compounds lower the apoptotic threshold of all cells that express anti-apoptotic Bcl-2 family proteins, the proportion of cells that successfully execute the apoptosis program in response to the apoptosis inducing activity of anticancer drugs/radiation is increased. Alternatively, the compounds of the present invention can be used to allow administration of a lower, and therefore less toxic and more tolerable, dose of an anticancer agent and/or radiation to produce the same tumor response/clinical benefit as the conventional dose of the anticancer agent/radiation alone. Since the doses for all approved anticancer drugs and radiation treatments are known, the present invention contemplates the various combinations of them with the present compounds. Also, since the compounds of the present invention act at least in part by inhibiting anti-apoptotic Bcl-2 family proteins, the exposure of cancer cells and supporting cells to therapeutically effective amounts of the compounds can be temporally linked to coincide with the attempts of cells to execute the apoptosis program in response to the anticancer agent or radiation therapy. Thus, in some embodiments, administering the compositions of the present invention in connection with certain temporal relationships provides especially efficacious therapeutic practices.

The present invention relates to apogossypolone (Formula I) or pharmaceutically acceptable salts or prodrugs thereof that are useful for inhibiting the activity of anti-apoptotic Bcl-2 family proteins, inhibiting hyperproliferation in cells, inducing apoptosis in cells, and increasing the sensitivity of cells to inducers of apoptosis.

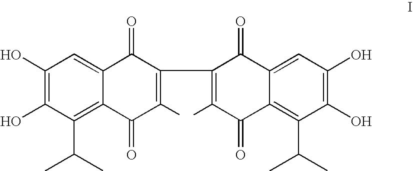

The compounds of the invention are useful for the treatment, amelioration, or prevention of disorders responsive to induction of apoptotic cell death, e.g., disorders characterized by dysregulation of apoptosis, including hyperproliferative diseases such as cancer. In certain embodiments, the compounds can be used to treat, ameliorate, or prevent cancer that is characterized by resistance to cancer therapies (e.g., those which are chemoresistant, radiation resistant, hormone resistant, and the like). In additional embodiments, the compounds can be used to treat, ameliorate, or prevent metastatic cancer. In other embodiments, the compounds can be used to treat hyperproliferative diseases characterized by overexpression of anti-apoptotic Bcl-2 family proteins.

Other compounds related to gossypol and apogossypolone may be useful for the treatment, amelioration, or prevention of disorders responsive to induction of apoptotic cell death, e.g., disorders characterized by dysregulation of apoptosis, including hyperproliferative diseases such as cancer. Such compounds include gossypolic acid (Formula V) and gossypolonic acid (Formula VI) or pharmaceutically acceptable salts or prodrugs thereof.

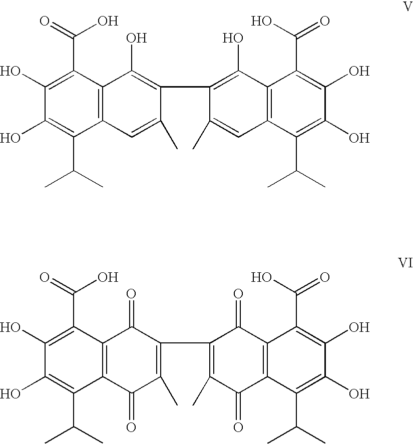

The present invention provides pharmaceutical compositions comprising compounds of the invention or pharmaceutically acceptable salts or prodrugs thereof in a therapeutically effective amount to inhibit hyperproliferation in cells, to induce apoptosis in cells or to sensitize cells to inducers of apoptosis.

The invention further provides kits comprising compounds of the invention or pharmaceutically acceptable salts or prodrugs thereof. The kits may optionally contain instructions for administering the compound to an animal and/or other therapeutic agents, e.g., anticancer agents.

The invention also provides methods of making compounds of the invention or pharmaceutically acceptable salts or prodrugs thereof. Also provided are compounds useful as intermediates in the synthesis of apogossypolone.

### BRIEF DESCRIPTION OF THE DRAWINGS/FIGURES

FIG. 1 shows the binding of apogossypolone to Bcl-2 and Bcl-xL.

FIG. 2 shows the inhibition of cell growth by apogossypolone and other gossypol derivatives in human breast cancer cell line MDA-MB-231 (subclone 2LMP) cells.

FIG. 3 shows the inhibition of cell growth by apogossypolone and other gossypol derivatives in human breast cancer cell line T47D cells.

FIG. 4 shows the inhibition of cell growth by apogossypolone and other gossypol derivatives in human breast cancer cell line MDA-435 cells.

FIG. 5 shows the inhibition of tumor growth by apogossypolone and X-ray radiation in a human prostate cancer cell line PC-3 xenograft nude mouse model.

FIG. 6 shows binding isotherms of different concentrations of the Flu-Bid-21mer peptide against Mcl-1 protein.

FIG. 7 shows competitive binding curves of unlabeled BID 21mer peptide, apogossypolone and (−)-gossypol against Mcl-1 as determined using a fluorescence-polarization-based binding assay.

### DETAILED DESCRIPTION OF THE INVENTION

The present invention relates to apogossypolone or pharmaceutically acceptable salts or prodrugs thereof, which function as inhibitors of anti-apoptotic Bcl-2 family proteins. By inhibiting anti-apoptotic Bcl-2 family proteins, apogossypolone inhibits hyperproliferation in cells, sensitizes cells to inducers of apoptosis and, in some instances, itself induces apoptosis. Therefore, the invention relates to methods of inhibiting hyperproliferation in cells, methods of sensitizing cells to inducers of apoptosis and methods of inducing apoptosis in cells, comprising contacting the cells with apogossypolone or salts or prodrugs thereof alone or in combination with an inducer of apoptosis. The invention further relates to methods of treating, ameliorating, or preventing disorders in an animal that are responsive to induction of apoptosis comprising administering to the animal apogossypolone or salts or prodrugs thereof and an inducer of apoptosis. Such disorders include those characterized by a dysregulation of apoptosis and those characterized by overexpression of anti-apoptotic Bcl-2 family proteins.

A further aspect of the invention relates to compounds related to gossypol or apogossypolone which also function as inhibitors of anti-apoptotic Bcl-2 family proteins and which may be used in the practice of the invention. Such compounds include gossypolic acid and gossypolonic acid or pharmaceutically acceptable salts or prodrugs thereof.

The term “Bcl-2 family proteins,” as used herein, refers to both the anti-apoptotic members of the Bcl-2 family, including, but not limited to, Bcl-2, Bcl-xL, Mcl-1, A1/BFL-1, BOO-DIVA, Bcl-w, Bcl-6, Bcl-8, and Bcl-y, and the pro-apoptotic members of the Bcl-2 family, including, but not limited to, Bak, Bax, Bad, tBid, Hrk, Bim, Bmf, as well as other Bcl-2 homology domain 3 (BH3) containing proteins that are regulated by apogossypolone compounds.

The term “overexpression of anti-apoptotic Bcl-2 family proteins,” as used herein, refers to an elevated level (e.g., aberrant level) of mRNAs encoding for an anti-apoptotic Bcl-2 family protein(s), and/or to elevated levels of anti-apoptotic Bcl-2 family protein(s) in cells as compared to similar corresponding non-pathological cells expressing basal levels of mRNAs encoding anti-apoptotic Bcl-2 family proteins or having basal levels of anti-apoptotic Bcl-2 family proteins. Methods for detecting the levels of mRNAs encoding anti-apoptotic Bcl-2 family proteins or levels of anti-apoptotic Bcl-2 family proteins in a cell include, but are not limited to, Western blotting using anti-apoptotic Bcl-2 family protein antibodies, immunohistochemical methods, and methods of nucleic acid amplification or direct RNA detection. As important as the absolute level of anti-apoptotic Bcl-2 family proteins in cells is to determining that they overexpress anti-apoptotic Bcl-2 family proteins, so also is the relative level of anti-apoptotic Bcl-2 family proteins to other pro-apoptotic signaling molecules (e.g., pro-apoptotic Bcl-2 family proteins) within such cells. When the balance of these two are such that, were it not for the levels of the anti-apoptotic Bcl-2 family proteins, the pro-apoptotic signaling molecules would be sufficient to cause the cells to execute the apoptosis program and die, said cells would be dependent on the anti-apoptotic Bcl-2 family proteins for their survival. In such cells, exposure to an inhibiting effective amount of an anti-apoptotic Bcl-2 family protein inhibitor is sufficient to cause the cells to execute the apoptosis program and die. Thus, the term “overexpression of an anti-apoptotic Bcl-2 family protein” also refers to cells that, due to the relative levels of pro-apoptotic signals and anti-apoptotic signals, undergo apoptosis in response to inhibiting effective amounts of compounds that inhibit the function of anti-apoptotic Bcl-2 family proteins.

The terms “anticancer agent” and “anticancer drug,” as used herein, refer to any therapeutic agents (e.g., chemotherapeutic compounds and/or molecular therapeutic compounds), radiation therapies, or surgical interventions, used in the treatment of hyperproliferative diseases such as cancer (e.g., in mammals).

The term “prodrug,” as used herein, refers to a pharmacologically inactive derivative of a parent “drug” molecule that requires biotransformation (e.g., either spontaneous or enzymatic) within the target physiological system to release, or to convert (e.g., enzymatically, mechanically, electromagnetically) the prodrug into the active drug. Prodrugs are designed to overcome problems associated with stability, toxicity, lack of specificity, or limited bioavailability. Exemplary prodrugs comprise an active drug molecule itself and a chemical masking group (e.g., a group that reversibly suppresses the activity of the drug). Some preferred prodrugs are variations or derivatives of compounds that have groups cleavable under metabolic conditions. Exemplary prodrugs become pharmaceutically active in vivo or in vitro when they undergo solvolysis under physiological conditions or undergo enzymatic degradation or other biochemical transformation (e.g., phosphorylation, hydrogenation, dehydrogenation, glycosylation). Prodrugs often offer advantages of solubility, tissue compatibility, or delayed release in the mammalian organism. (See e.g., Bundgard, Design of Prodrugs, pp. 7-9, 21-24, Elsevier, Amsterdam (1985); and Silverman, The Organic Chemistry of Drug Design and Drug Action, pp. 352-401, Academic Press, San Diego, Calif. (1992)). Common prodrugs include acid derivatives such as esters prepared by reaction of the hydroxyl groups of apogossypolone with a suitable carboxylic acid (e.g., a lower carboxylic acid such as acetic acid), and imines prepared by reaction of the ketone groups of apogossypolone with an amine (e.g., a lower primary or secondary alkylamine).

The term “pharmaceutically acceptable salt,” as used herein, refers to any salt (e.g., obtained by reaction with an acid or a base) of a compound of the present invention that is physiologically tolerated in the target animal (e.g., a mammal). Salts of the compounds of the present invention may be derived from inorganic or organic bases. Examples of bases include, but are not limited to, alkali metal (e.g., sodium and lithium) hydroxides, alkaline earth metal (e.g., magnesium) hydroxides, ammonia, and compounds of Formula NW₄⁺, wherein W is C₁₋₄ alkyl, and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of cancer, a therapeutically effective amount preferably refers to the amount of a therapeutic agent that decreases the rate of tumor growth, decreases tumor mass, decreases the number of metastases, increases time to tumor progression, or increases survival time by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The terms “sensitize” and “sensitizing,” as used herein, refer to making, through the administration of a first agent (e.g., a compound of Formula I), an animal or a cell within an animal more susceptible, or more responsive, to the biological effects (e.g., promotion or retardation of an aspect of cellular function including, but not limited to, cell growth, proliferation, invasion, angiogenesis, or apoptosis) of a second agent. The sensitizing effect of a first agent on a target cell can be measured as the difference in the intended biological effect (e.g., promotion or retardation of an aspect of cellular function including, but not limited to, cell growth, proliferation, invasion, angiogenesis, or apoptosis) observed upon the administration of a second agent with and without administration of the first agent. The response of the sensitized cell can be increased by at least 10%, at least 20%, at least 30%, at least 40%, at least 50%, at least 60%, at least 70%, at least 80%, at least 90%, at least 100%, at least 150%, at least 200%, at least 350%, at least 300%, at least 350%, at least 400%, at least 450%, or at least 500% over the response in the absence of the first agent.

The term “dysregulation of apoptosis,” as used herein, refers to any aberration in the ability of (e.g., predisposition) a cell to undergo cell death via apoptosis. Dysregulation of apoptosis is associated with or induced by a variety of conditions, including for example, autoimmune disorders (e.g., systemic lupus erythematosus, rheumatoid arthritis, graft-versus-host disease, myasthenia gravis, or Sjögren's syndrome), chronic inflammatory conditions (e.g., psoriasis, asthma, or Crohn's disease), hyperproliferative disorders (e.g., tumors, B cell lymphomas, or T cell lymphomas), viral infections (e.g., herpes, papilloma, or HIV), and other conditions such as osteoarthritis and atherosclerosis. It should be noted that when the dysregulation is induced by or associated with a viral infection, the viral infection may or may not be detectable at the time dysregulation occurs or is observed. That is, viral-induced dysregulation can occur even after the disappearance of symptoms of viral infection.

The term “hyperproliferative disease,” as used herein, refers to any condition in which a localized population of proliferating cells in an animal is not governed by the usual limitations of normal growth. Examples of hyperproliferative disorders include tumors, neoplasms, lymphomas, and the like. A neoplasm is said to be benign if it does not undergo invasion or metastasis and malignant if it does either of these. A “metastatic” cell means that the cell can invade and destroy neighboring body structures. Hyperplasia is a form of cell proliferation involving an increase in cell number in a tissue or organ without significant alteration in structure or function. Metaplasia is a form of controlled cell growth in which one type of fully differentiated cell substitutes for another type of differentiated cell.

The pathological growth of activated lymphoid cells often results in an autoimmune disorder or a chronic inflammatory condition. As used herein, the term “autoimmune disorder” refers to any condition in which an organism produces antibodies or immune cells which recognize the organism's own molecules, cells or tissues. Non-limiting examples of autoimmune disorders include autoimmune hemolytic anemia, autoimmune hepatitis, Berger's disease or IgA nephropathy, celiac sprue, chronic fatigue syndrome, Crohn's disease, dermatomyositis, fibromyalgia, graft versus host disease, Grave's disease, Hashimoto's thyroiditis, idiopathic thrombocytopenia purpura, lichen planus, multiple sclerosis, myasthenia gravis, psoriasis, rheumatic fever, rheumatic arthritis, scleroderma, Sjögren's syndrome, systemic lupus erythematosus, type 1 diabetes, ulcerative colitis, vitiligo, and the like.

The term “neoplastic disease,” as used herein, refers to any abnormal growth of cells being either benign (non-cancerous) or malignant (cancerous).

The term “anti-neoplastic agent,” as used herein, refers to any compound that retards the proliferation, growth, or spread of a targeted (e.g., malignant) neoplasm.

The terms “prevent,” “preventing,” and “prevention,” as used herein, refer to a decrease in the occurrence of pathological cells (e.g., hyperproliferative or neoplastic cells) in an animal. The prevention may be complete, e.g., the total absence of pathological cells in a subject. The prevention may also be partial, such that the occurrence of pathological cells in a subject is less than that which would have occurred without the present invention.

The term “synergistic,” as used herein, refers to an effect obtained when apogossypolone and a second agent are administered together (e.g., at the same time or one after the other) that is greater than the additive effect of apogossypolone and the second agent when administered individually. The synergistic effect allows for lower doses of apogossypolone and/or the second agent to be administered or provides greater efficacy at the same doses. The synergistic effect obtained can be at least 10%, at least 20%, at least 30%, at least 40%, at least 50%, at least 60%, at least 70%, at least 80%, at least 90%, at least 100%, at least 125%, at least 150%, at least 175%, at least 200%, at least 250%, at least 300%, at least 350%, at least 400%, or at least 500% more than the additive effect of the apogossypolone compound and the second agent when administered individually. For example, with respect to the treatment of cancer, the synergistic effect can be a decrease in the rate of tumor growth, a decrease in tumor mass, a decrease in the number of metastases, an increase in time to tumor progression, or an increase in survival time. As described herein, apogossypolone compounds and anticancer agents, when administered individually, often only inhibit tumor cell proliferation rather than cause regression of the tumor mass. According to the present invention, administration of apogossypolone compounds and anticancer agents is used to cause an actual regression of tumor mass. The co-administration of apogossypolone and an anticancer agent may allow for the use of lower doses of apogossypolone and/or the anticancer agent such that the cancer is effectively treated while avoiding any substantial toxicity to the subject.

The inhibitors of anti-apoptotic Bcl-2 family proteins of the present invention include apogossypolone (Formula I) or pharmaceutically acceptable salts or prodrugs thereof.

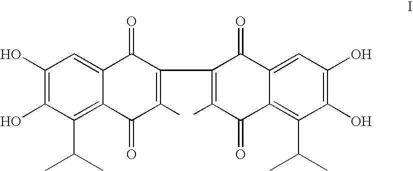

Other inhibitors of anti-apoptotic Bcl-2 family proteins of the present invention include gossypolic acid (Formula V) and gossypolonic acid (Formula VI) or pharmaceutically acceptable salts or prodrugs thereof.

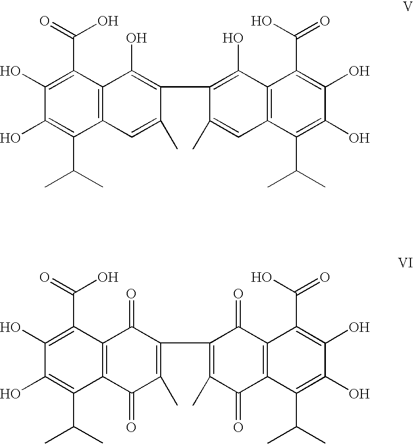

Certain of the compounds of the present invention may exist as stereoisomers including optical isomers, e.g., (+)-apogossypolone, (−)-apogossypolone, (+)-gossypolic acid, (−)-gossypolic acid, (+)-gossypolonic acid, and (−)-gossypolonic acid. Preferably, the (+)-apogossypolone, (−)-apogossypolone, (+)-gossypolic acid, (−)-gossypolic acid, (+)-gossypolonic acid, and (−)-gossypolonic acid each have an enantiomeric excess of 1% to 100%. In one embodiment, the (+)-apogossypolone, (−)-apogossypolone, (+)-gossypolic acid, (−)-gossypolic acid, (+)-gossypolonic acid, and (−)-gossypolonic acid each have an enantiomeric excess of at least 10%, 20%, 30%, 40%, 50%, 60%, 70%, 80%, 90%, 91%, 92%, 93%, 94%, 95%, 96%, 97%, 98%, 99%, or 100%. The invention includes all stereoisomers and both the racemic mixtures of such stereoisomers as well as the individual enantiomers that may be separated according to methods that are well known to those of skill in the art.

The compounds of this invention may be prepared using methods known to those of skill in the art and as disclosed in the Examples. In one embodiment, apogossypolone is synthesized from gossypol by the method shown in Scheme I, wherein P is a protecting group. In an alternative embodiment, a chiral HPLC column may be used to separate (±)-apogossypolone into its (+) and (−) enantiomers.

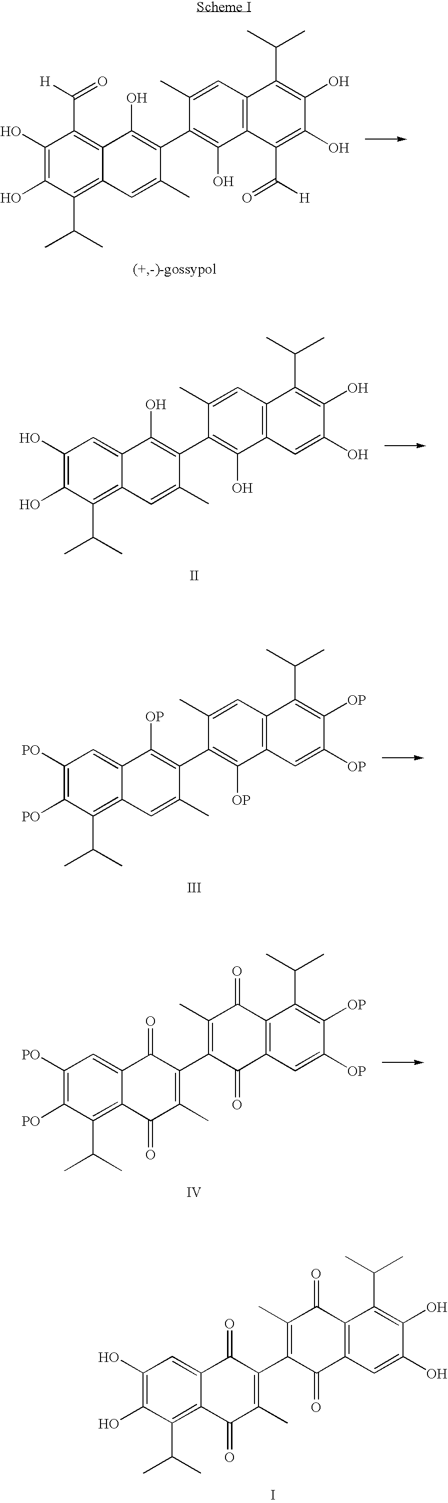

In one embodiment, gossypolonic acid is synthesized from gossypol by the method shown in Scheme II.

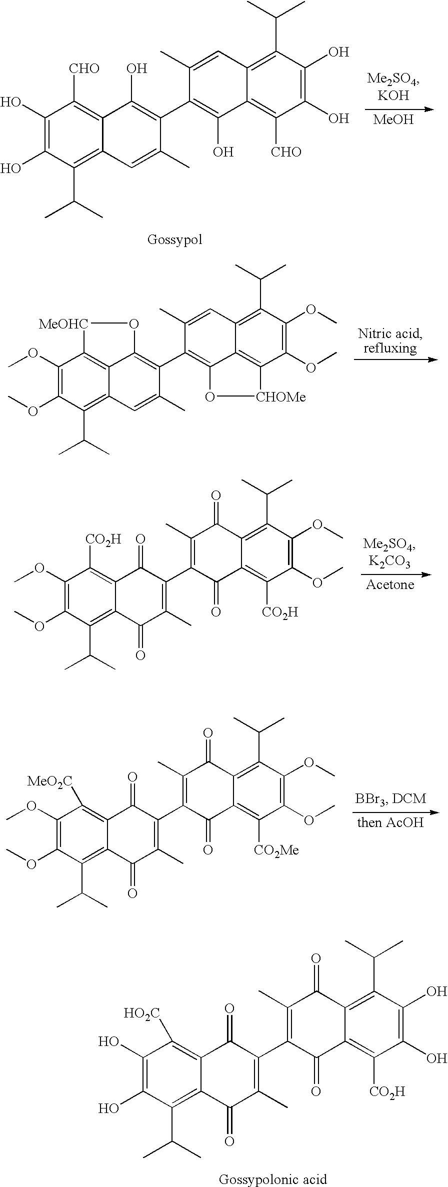

The invention also relates to a method of preparing apogossypolone, comprising
(a) decarbonylating gossypol to give a compound of Formula II:

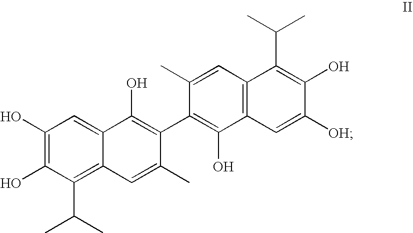

(b) protecting the hydroxy groups of the compound of Formula II to give a compound of Formula III, wherein P is a protecting group:

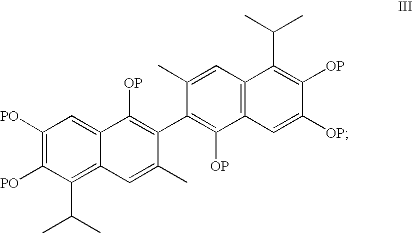

(c) oxidizing the compound of Formula III to give a compound of Formula IV:

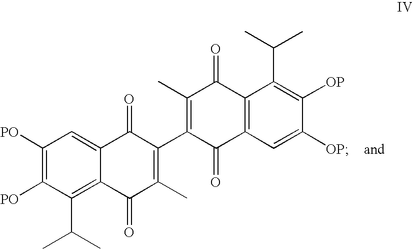

(d) deprotecting the compound of Formula IV to give apogossypolone.

Gossypol may be decarbonylated by heating gossypol in a solvent under basic conditions. Gossypol may also be decarbonylated by reaction with HSCH₂CH₂SH in the presence of BF₃/Et₂O. For example, gossypol may be heated in aqueous NaOH or KOH at about 40 to 150° C., more preferably about 85° C. The reaction is carried out preferably under an inert atmosphere (e.g., argon or nitrogen).

Protecting groups “P” include any suitable protecting group, such as lower alkanoyl, aralkanoyl, benzoyl, and alkyl/aryl silyl groups, e.g., t-butyldimethyl silyl groups. Examples of alkanoyl groups include acetyl, propionyl, t-butanoyl, and the like. Examples of aralkanoyl groups include phenylacetyl and 1-phenyl-1-methyl acetyl groups.

The hydroxy protected compound of Formula III may be prepared by reacting the compound of Formula II with a suitable reagent such as the anhydride or acid halide of the corresponding alkanoic, aralkanoic, or benzoic acid. Where the protecting group is an alkyl or alkyl/aryl silyl group, the corresponding silyl chloride reagent may be used. The reaction is carried out in a suitable aprotic solvent at ambient temperature for up to 12 hours in the presence of an organic base such as N,N′-diisopropylethylamine, pyridine, or dimethylaminopyridiine.

The compound of Formula IV may be prepared by oxidizing the compound of Formula III with a suitable oxidizing reagent such as periodic acid or chromium (VI) oxide, in a suitable solvent such as dioxane, acetonitrile or acetic acid at about 40 to 150° C., preferably at about 80 to 120° C. for 10 to 600 minutes. Isolation of the compound of Formula IV may be achieved by any conventional method such as extraction and chromatography.

Apogossypolone may then be prepared by removal of the protecting groups of the compound of Formula IV. Where the protecting groups are alkanoyl, aralkanoyl, or benzoyl groups, they may be removed by reacting the compound of Formula IV with a base in an appropriate solvent. Examples of bases include sodium and potassium carbonate and lithium, sodium, and potassium hydroxide and appropriate solvents include ethers such as dioxane and polar non-protic solvents such as dimethylformamide and dimethylsulfoxide. The apogossypolone may then be acidified and isolated by extraction and then purified by crystallization/recrystallization to give purified apogossypolone.

The invention also relates to compounds useful as intermediates in the synthesis of apogossypolone, including compounds of Formulas II, III, and IV.

An important aspect of the present invention is that apogossypolone binds to and inhibits anti-apoptotic Bcl-2 proteins in the same manner as gossypol. However, apogossypolone binds more tightly and is a more potent inhibitor than gossypol while having less toxicity. Thus, apogossypolone or pharmaceutically acceptable salts or prodrugs thereof inhibit hyperproliferation, induce apoptosis and also potentiate the induction of apoptosis in response to apoptosis induction signals. It is contemplated that these compounds sensitize cells to inducers of apoptosis, including cells that are resistant to such inducers. The anti-apoptotic Bcl-2 family protein inhibitors of the present invention can be used to induce apoptosis in any disorder that can be treated, ameliorated, or prevented by the induction of apoptosis. Thus, the present invention provides compositions and methods for targeting animals characterized as overexpressing an anti-apoptotic Bcl-2 family protein. In some of the embodiments, the cells (e.g., cancer cells) show elevated expression levels of one or more anti-apoptotic Bcl-2 family proteins as compared to non-pathological samples (e.g., non-cancerous cells). In other embodiments, the cells operationally manifest elevated expression levels of anti-apoptotic Bcl-2 family proteins by virtue of executing the apoptosis program and dying in response to an inhibiting effective amount of apogossypolone, said response occurring, at least in part, due to the dependence in such cells on anti-apoptotic Bcl-2 family protein function for their survival.

In some embodiments, the compositions and methods of the present invention are used to treat diseased cells, tissues, organs, or pathological conditions and/or disease states in an animal (e.g., a mammalian subject including, but not limited to, humans and veterinary animals). In this regard, various diseases and pathologies are amenable to treatment or prophylaxis using the present methods and compositions. A non-limiting exemplary list of these diseases and conditions includes, but is not limited to, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, and the like, T and B cell mediated autoimmune diseases, inflammatory diseases, infections, hyperproliferative diseases, AIDS, degenerative conditions, vascular diseases, and the like. In some embodiments, the cancer cells being treated are metastatic. In other embodiments, the cancer cells being treated are resistant to anticancer agents.

In some embodiments, infections suitable for treatment with the compositions and methods of the present invention include, but are not limited to, infections caused by viruses, bacteria, fungi, mycoplasma, prions, and the like.

Some embodiments of the present invention provide methods for administering an effective amount of apogossypolone and at least one additional therapeutic agent (including, but not limited to, chemotherapeutic antineoplastics, antimicrobials, antivirals, antifungals, and anti-inflammatory agents) and/or therapeutic technique (e.g., surgical intervention, and/or radiotherapies). In some embodiments, the combination of apogossypolone and one or more therapeutic agents is expected to have a greater effect as compared to the administration of either compound alone. In other embodiments, the combination of apogossypolone and one or more therapeutic agents is expected to result in a synergistic effect (i.e., more than additive) as compared to the administration of either one alone.

A number of suitable anticancer agents are contemplated for use in the methods of the present invention. Indeed, the present invention contemplates, but is not limited to, administration of numerous anticancer agents such as: agents that induce apoptosis; polynucleotides (e.g., anti-sense, ribozymes, siRNA); polypeptides (e.g., enzymes and antibodies); biological mimetics (e.g., gossypol or BH3 mimetics); agents that bind (e.g., oligomerize or complex) with a Bcl-2 family protein such as Bax; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of chemotherapeutic compounds and anticancer therapies suitable for co-administration with the disclosed compounds are known to those skilled in the art.

In preferred embodiments, anticancer agents comprise agents that induce or stimulate apoptosis. Agents that induce apoptosis include, but are not limited to, radiation (e.g., X-rays, gamma rays, UV); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine, dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the compositions and methods of the present invention provide a compound of Formula I and at least one anti-hyperproliferative or antineoplastic agent selected from alkylating agents, antimetabolites, and natural products (e.g., herbs and other plant and/or animal derived compounds).

Alkylating agents suitable for use in the present compositions and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil), floxuridine (fluorode-oxyuridine), and cytarabine (cytosine arabinoside)); and 3) purine analogs (e.g., mercaptopurine (6-mercaptopurine), thioguanine (6-thioguanine), and pentostatin (2′-deoxycoformycin)).

In still further embodiments, chemotherapeutic agents suitable for use in the compositions and methods of the present invention include, but are not limited to: 1) vinca alkaloids (e.g., vinblastine, vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin and carboplatin); 7) anthracenediones (e.g., mitoxantrone); 8) substituted ureas (e.g., hydroxyurea); 9) methylhydrazine derivatives (e.g., procarbazine (N-methylhydrazine)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table 1 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 1

| | | |
|---|---|---|
| Aldesleukin | Proleukin | Chiron Corp., |
| (des-alanyl-1, serine-125 human interleukin-2) | | Emeryville, CA |
| Alemtuzumab | Campath | Millennium and ILEX |
| (IgG1κ anti CD52 antibody) | | Partners, LP, |
| | | Cambridge, MA |
| Alitretinoin | Panretin | Ligand |
| (9-cis-retinoic acid) | | Pharmaceuticals, Inc., |
| | | San Diego CA |
| Allopurinol | Zyloprim | GlaxoSmithKline, |
| (1,5-dihydro-4 H-pyrazolo[3,4-d]pyrimidin-4-one | | Research Triangle |
| monosodium salt) | | Park, NC |
| Altretamine | Hexalen | US Bioscience, West |
| (N,N,N′,N′,N″,N″,-hexamethyl-1,3,5-triazine-2,4, | | Conshohocken, PA |
| 6-triamine) | | |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca |
| (1,3-Benzenediacetonitrile,a,a,a′,a′-tetramethyl- | | Pharmaceuticals, LP, |
| 5-(1H-1,2,4-triazol-1-ylmethyl)) | | Wilmington, DE |
| Arsenic trioxide | Trisenox | Cell Therapeutic, Inc., |
| | | Seattle, WA |
| Asparaginase | Elspar | Merck &Co., Inc., |
| (L-asparagine amidohydrolase, type EC-2) | | Whitehouse Station, |
| | | NJ |
| BCG Live | TICE BCG | Organon Teknika, |
| (lyophilized preparation of an attenuated strain of | | Corp., Durham, NC |
| Mycobacterium bovis (Bacillus Calmette-Gukin | | |
| [BCG], substrain Montreal) | | |
| bexarotene capsules | Targretin | Ligand |
| (4-[1-(5,6,7,8-tetrahydro-3,5,5,8,8-pentamethyl-2- | | Pharmaceuticals |
| napthalenyl) ethenyl] benzoic acid) | | |
| bexarotene gel | Targretin | Ligand |
| | | Pharmaceuticals |
| Bleomycin | Blenoxane | Bristol-Myers Squibb |
| (cytotoxic glycopeptide antibiotics produced by | | Co., NY, NY |
| Streptomyces verticillus; bleomycin A₂ and | | |
| bleomycin B₂) | | |
| Capecitabine | Xeloda | Roche |
| (5′-deoxy-5-fluoro-N-[(pentyloxy)carbonyl]- | | |
| cytidine) | | |
| Carboplatin | Paraplatin | Bristol-Myers Squibb |
| (platinum, diammine [1,1- | | |
| cyclobutanedicarboxylato(2-)-0,0′]-,(SP-4-2)) | | |
| Carmustine | BCNU, BiCNU | Bristol-Myers Squibb |
| (1,3-bis(2-chloroethyl)-1-nitrosourea) | | |
| Carmustine with Polifeprosan 20 Implant | Gliadel Wafer | Guilford |
| | | Pharmaceuticals, Inc., |
| | | Baltimore, MD |
| Celecoxib | Celebrex | Searle |
| (as 4-[5-(4-methylphenyl)-3-(trifluoromethyl)-1H- | | Pharmaceuticals, |
| pyrazol-1-yl] benzenesulfonamide) | | England |
| Chlorambucil | Leukeran | GlaxoSmithKline |
| (4-[bis(2chlorethyl)amino]benzenebutanoic acid) | | |
| Cisplatin | Platinol | Bristol-Myers Squibb |
| (PtCl₂H₆N₂) | | |
| Cladribine | Leustatin, 2-CdA | R. W. Johnson |
| (2-chloro-2′-deoxy-b-D-adenosine) | | Pharmaceutical |
| | | Research Institute, |
| | | Raritan, NJ |
| Cyclophosphamide | Cytoxan, Neosar | Bristol-Myers Squibb |
| (2-[bis(2-chloroethyl)amino] tetrahydro-2H-13,2- | | |
| oxazaphosphorine 2-oxide monohydrate) | | |
| Cytarabine | Cytosar-U | Pharmacia &Upjohn |
| (1-b-D-Arabinofuranosylcytosine, C₉H₁₃N₃O₅) | | Company |
| cytarabine liposomal | DepoCyt | Skye |
| | | Pharmaceuticals, Inc., |
| | | San Diego, CA |
| Dacarbazine | DTIC-Dome | Bayer AG, |
| (5-(3,3-dimethyl-1-triazeno)-imidazole-4- | | Leverkusen, Germany |
| carboxamide (DTIC)) | | |
| Dactinomycin, actinomycin D | Cosmegen | Merck |
| (actinomycin produced by Streptomyces parvullus, | | |
| C₆₂H₈₆N₁₂O₁₆) | | |
| Darbepoetin alfa | Aranesp | Amgen, Inc., |
| (recombinant peptide) | | Thousand Oaks, CA |
| daunorubicin liposomal | DanuoXome | Nexstar |
| ((8S-cis)-8-acetyl-10-[(3-amino-2,3,6-trideoxy-á- | | Pharmaceuticals, Inc., |
| L-lyxo-hexopyranosyl)oxy]-7,8,9,10-tetrahydro- | | Boulder, CO |
| 6,8,11-trihydroxy-1-methoxy-5,12- | | |
| naphthacenedione hydrochloride) | | |
| Daunorubicin HCl, daunomycin | Cerubidine | Wyeth Ayerst, |
| ((1 S,3 S)-3-Acetyl-1,2,3,4,6,11-hexahydro- | | Madison, NJ |
| 3,5,12-trihydroxy-10-methoxy-6,11-dioxo-1- | | |
| naphthacenyl 3-amino-2,3,6-trideoxy-(alpha)-L- | | |
| lyxo-hexopyranoside hydrochloride) | | |
| Denileukin diftitox | Ontak | Seragen, Inc., |
| (recombinant peptide) | | Hopkinton, MA |
| Dexrazoxane | Zinecard | Pharmacia &Upjohn |
| ((S)-4,4′-(1-methyl-1,2-ethanediyl)bis-2,6- | | Company |
| piperazinedione) | | |
| Docetaxel | Taxotere | Aventis |
| ((2R,3S)-N-carboxy-3-phenylisoserine, N-tert- | | Pharmaceuticals, Inc., |
| butyl ester, 13-ester with 5b-20-epoxy- | | Bridgewater, NJ |
| 12a,4,7b,10b,13a-hexahydroxytax-11-en-9-one 4- | | |
| acetate 2-benzoate, trihydrate) | | |
| Doxorubicin HCl | Adriamycin, | Pharmacia &Upjohn |
| (8S,10S)-10-[(3-amino-2,3,6-trideoxy-a-L-lyxo- | Rubex | Company |
| hexopyranosyl)oxy]-8-glycolyl-7,8,9,10- | | |
| tetrahydro-6,8,11-trihydroxy-1-methoxy-5,12- | | |
| naphthacenedione hydrochloride) | | |
| doxorubicin | Adriamycin PFS | Pharmacia &Upjohn |
| | Intravenous | Company |
| | injection | |
| doxorubicin liposomal | Doxil | Sequus |
| | | Pharmaceuticals, Inc., |
| | | Menlo park, CA |
| dromostanolone propionate | Dromostanolone | Eli Lilly &Company, |
| (17b-Hydroxy-2a-methyl-5a-androstan-3-one propionate) | | Indianapolis, IN |
| dromostanolone propionate | Masterone | Syntex, Corp., Palo |
| | injection | Alto, CA |
| Elliott's B Solution | Elliott's B | Orphan Medical, Inc |
| | Solution | |
| Epirubicin | Ellence | Pharmacia &Upjohn |
| ((8S-cis)-10-[(3-amino-2,3,6-trideoxy-a-L-arabino- | | Company |
| hexopyranosyl)oxy]-7,8,9,10-tetrahydro-6,8,11- | | |
| trihydroxy-8-(hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia &Upjohn |
| (estra-1,3,5(10)-triene-3,17-diol(17(beta))-, 3- | | Company |
| [bis(2-chloroethyl)carbamate] 17-(dihydrogen | | |
| phosphate), disodium salt, monohydrate, or | | |
| estradiol 3-[bis(2-chloroethyl)carbamate] 17- | | |
| (dihydrogen phosphate), disodium salt, | | |
| monohydrate) | | |
| Etoposide phosphate | Etopophos | Bristol-Myers Squibb |
| (4′-Demethylepipodophyllotoxin 9-[4,6-O—(R)- | | |
| ethylidene-(beta)-D-glucopyranoside], | | |
| 4′-(dihydrogen phosphate)) | | |
| etoposide, VP-16 | Vepesid | Bristol-Myers Squibb |
| (4′-demethylepipodophyllotoxin 9-[4,6-0-(R)- | | |
| ethylidene-(beta)-D-glucopyranoside]) | | |
| Exemestane | Aromasin | Pharmacia &Upjohn |
| (6-methylenandrosta-1,4-diene-3, 17-dione) | | Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, |
| (fluorinated nucleotide analog of the antiviral agent | | Inc., Cedar Knolls, NJ |
| vidarabine, 9-b-D-arabinofuranosyladenine (ara- | | |
| A)) | | |
| Fluorouracil, 5-FU | Adrucil | ICN Pharmaceuticals, |
| (5-fluoro-2,4(1H,3H)-pyrimidinedione) | | Inc., Humacao, Puerto |
| | | Rico |
| Fulvestrant | Faslodex | IPR Pharmaceuticals, |
| (7-alpha-[9-(4,4,5,5,5-penta fluoropentylsulphinyl) | | Guayama, Puerto |
| nonyl]estra-1,3,5-(10)-triene-3,17-beta-diol) | | Rico |
| Gemcitabine | Gemzar | Eli Lilly |
| (2′-deoxy-2′,2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex Implant | AstraZeneca |
| (acetate salt of [D-Ser(But)⁶,Azgly¹⁰]LHRH; pyro- | | Pharmaceuticals |
| Glu-His-Trp-Ser-Tyr-D-Ser(But)-Leu-Arg-Pro- | | |
| Azgly-NH2 acetate [C₅₉H₈₄N₁₈O₁₄ •(C₂H₄O₂)ₓ | | |
| Hydroxyurea | Hydrea | Bristol-Myers Squibb |
| Ibritumomab Tiuxetan | Zevalin | Biogen IDEC, Inc., |
| (immunoconjugate resulting from a thiourea | | Cambridge MA |
| covalent bond between the monoclonal antibody | | |
| Ibritumomab and the linker-chelator tiuxetan [N- | | |
| [2-bis(carboxymethyl)amino]-3-(p- | | |
| isothiocyanatophenyl)-propyl]-[N-[2- | | |
| bis(carboxymethyl)amino]-2-(methyl)- | | |
| ethyl]glycine) | | |
| Idarubicin | Idamycin | Pharmacia &Upjohn |
| (5,12-Naphthacenedione, 9-acetyl-7-[(3-amino- | | Company |
| 2,3,6-trideoxy-(alpha)-L-lyxo- | | |
| hexopyranosyl)oxy]-7,8,9,10-tetrahydro-6,9,11- | | |
| trihydroxyhydrochloride,(7S-cis)) | | |
| Ifosfamide | IFEX | Bristol-Myers Squibb |
| (3-(2-chloroethyl)-2-[(2- | | |
| chloroethyl)amino]tetrahydro-2H-1,3,2- | | |
| oxazaphosphorine 2-oxide) | | |
| Imatinib Mesilate | Gleevec | Novartis AG, Basel, |
| (4-[(4-Methyl-1-piperazinyl)methyl]-N-[4-methyl- | | Switzerland |
| 3-[[4-(3-pyridinyl)-2-pyrimidinyl]amino]- | | |
| phenyl]benzamide methanesulfonate) | | |
| Interferon alfa-2a | Roferon-A | Hoffmann-La Roche, |
| (recombinant peptide) | | Inc., Nutley, NJ |
| Interferon alfa-2b | Intron A | Schering AG, Berlin, |
| recombinant peptide) | (Lyophilized | Germany |
| | Betaseron) | |
| Irinotecan HCl | Camptosar | Pharmacia &Upjohn |
| ((4S)-4,11-diethyl-4-hydroxy-9-[(4-piperi- | | Company |
| dinopiperidino)carbonyloxy]-1H-pyrano[3′,4′:6,7] | | |
| indolizino[1,2-b] quinoline-3,14(4H,12H) dione | | |
| hydrochloride trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., |
| (L-Glutamic acid, N[4[[(2amino-5-formyl- | Leucovorin | Seattle, WA |
| 1,4,5,6,7,8 hexahydro4oxo6- | | |
| pteridinyl)methyl]amino]benzoyl], calcium salt | | |
| (1:1)) | | |
| Levamisole HCl | Ergamisol | Janssen Research |
| ((−)-(S)-2,3,5,6-tetrahydro-6-phenylimidazo [2,1- | | Foundation, |
| b] thiazole monohydrochloride C₁₁H₁₂N₂S•HCl) | | Titusville, NJ |
| Lomustine | CeeNU | Bristol-Myers Squibb |
| (1-(2-chloro-ethyl)-3-cyclohexyl-1-nitrosourea) | | |
| Meclorethamine, nitrogen mustard | Mustargen | Merck |
| (2-chloro-N-(2-chloroethyl)-N-methylethanamine | | |
| hydrochloride) | | |
| Megestrol acetate | Megace | Bristol-Myers Squibb |
| 17α(acetyloxy)-6-methylpregna-4,6-diene- | | |
| 3,20-dione | | |
| Melphalan, L-PAM | Alkeran | GlaxoSmithKline |
| (4-[bis(2-chloroethyl) amino]-L-phenylalanine) | | |
| Mercaptopurine, 6-MP | Purinethol | GlaxoSmithKline |
| (1,7-dihydro-6H-purine-6-thione monohydrate) | | |
| Mesna | Mesnex | Asta Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L- | | |
| glutamic acid) | | |
| Methoxsalen | Uvadex | Therakos, Inc., Way |
| (9-methoxy-7H-furo[3,2-g][1]-benzopyran-7-one) | | Exton, Pa |
| Mitomycin C | Mutamycin | Bristol-Myers Squibb |
| mitomycin C | Mitozytrex | SuperGen, Inc., |
| | | Dublin, CA |
| Mitotane | Lysodren | Bristol-Myers Squibb |
| (1,1-dichloro-2-(o-chlorophenyl)-2-(p- | | |
| chlorophenyl) ethane) | | |
| Mitoxantrone | Novantrone | Immunex Corporation |
| (1,4-dihydroxy-5,8-bis[[2-[(2- | | |
| hydroxyethyl)amino]ethyl]amino]-9,10- | | |
| anthracenedione dihydrochloride) | | |
| Nandrolone phenpropionate | Durabolin-50 | Organon, Inc., West |
| | | Orange, NJ |
| Nofetumomab | Verluma | Boehringer Ingelheim |
| | | Pharma KG, Germany |
| Oprelvekin | Neumega | Genetics Institute, |
| (IL-11) | | Inc., Alexandria, VA |
| Oxaliplatin | Eloxatin | Sanofi Synthelabo, |
| (cis-[(1R,2R)-1,2-cyclohexanediamine-N,N′] | | Inc., NY, NY |
| [oxalato(2-)-O,O′] platinum) | | |
| Paclitaxel | TAXOL | Bristol-Myers Squibb |
| (5β,20-Epoxy-1,2a,4,7β,10β,13a- | | |
| hexahydroxytax-11-en-9-one 4,10-diacetate 2- | | |
| benzoate 13-ester with (2R,3S)-N-benzoyl-3- | | |
| phenylisoserine) | | |
| Pamidronate | Aredia | Novartis |
| (phosphonic acid (3-amino-1-hydroxypropylidene) | | |
| bis-, disodium salt, pentahydrate, (APD)) | | |
| Pegademase | Adagen | Enzon |
| ((monomethoxypolyethylene glycol succinimidyl) | (Pegademase | Pharmaceuticals, Inc., |
| 11-17-adenosine deaminase) | Bovine) | Bridgewater, NJ |
| Pegaspargase | Oncaspar | Enzon |
| (monomethoxypolyethylene glycol succinimidyl L- | | |
| asparaginase) | | |
| Pegfilgrastim | Neulasta | Amgen, Inc |
| (covalent conjugate of recombinant methionyl | | |
| human G-CSF (Filgrastim) and | | |
| monomethoxypolyethylene glycol) | | |
| Pentostatin | Nipent | Parke-Davis |
| | | Pharmaceutical Co., |
| | | Rockville, MD |
| Pipobroman | Vercyte | Abbott Laboratories, |
| | | Abbott Park, IL |
| Plicamycin, Mithramycin | Mithracin | Pfizer, Inc., NY, NY |
| (antibiotic produced by Streptomyces plicatus) | | |
| Porfimer sodium | Photofrin | QLT Phototherapeutics, |
| | | Inc., Vancouver, |
| | | Canada |
| Procarbazine | Matulane | Sigma Tau |
| N-isopropyl-μ-(2-methylhydrazino)-p-toluamide | | Pharmaceuticals, Inc., |
| monohydrochloride) | | Gaithersburg, MD |
| Quinacrine | Atabrine | Abbott Labs |
| 6-chloro-9-(1-methyl-4-diethyl-amine) | | |
| butylamino-2-methoxyacridine) | | |
| Rasburicase | Elitek | Sanofi-Synthelabo, |
| recombinant peptide) | | Inc., |
| Rituximab | Rituxan | Genentech, Inc., |
| (recombinant anti-CD20 antibody) | | South San Francisco, |
| | | CA |
| Sargramostim | Prokine | Immunex Corp |
| recombinant peptide) | | |
| Streptozocin | Zanosar | Pharmacia &Upjohn |
| (streptozocin 2-deoxy-2- | | Company |
| [[(methylnitrosoamino)carbonyl]amino]-a(and b)- | | |
| D-glucopyranose and 220 mg citric acid | | |
| anhydrous) | | |
| Talc | Sclerosol | Bryan, Corp., |
| (Mg₃Si₄O₁₀ (OH)₂) | | Woburn, MA |
| Tamoxifen | Nolvadex | AstraZeneca |
| ((Z)2-[4-(1,2-diphenyl-1-butenyl) phenoxy]-N,N- | | Pharmaceuticals |
| dimethylethanamine 2-hydroxy-1,2,3 - | | |
| propanetricarboxylate (1:1)) | | |
| Temozolomide | Temodar | Schering |
| (3,4-dihydro-3-methyl-4-oxoimidazo[5,1-d]-as- | | |
| tetrazine-8-carboxamide) | | |
| teniposide, VM-26 | Vumon | Bristol-Myers Squibb |
| (4′-demethylepipodophyllotoxin 9-[4,6-0-(R)-2- | | |
| thenylidene-(beta)-D-glucopyranoside]) | | |
| Testolactone | Teslac | Bristol-Myers Squibb |
| (13-hydroxy-3-oxo-13,17-secoandrosta-1,4-dien- | | |
| 17-oic acid [dgr]-lactone) | | |
| Thioguanine, 6-TG | Thioguanine | GlaxoSmithKline |
| (2-amino-1,7-dihydro-6H-purine-6-thione) | | |
| Thiotepa | Thioplex | Immunex Corporation |
| (Aziridine, 1.1′,1″-phosphinothioylidynetris-, or | | |
| Tris (1-aziridinyl) phosphine sulfide) | | |
| Topotecan HCl | Hycamtin | GlaxoSmithKline |
| ((S)-10-[(dimethylamino) methyl]-4-ethyl-4,9- | | |
| dihydroxy-1H-pyrano[3′,4′: 6,7] indolizino [1,2-b] | | |
| quinoline-3,14-(4H,12H)-dione | | |
| monohydrochloride) | | |
| Toremifene | Fareston | Roberts |
| (2-(p-[(Z)-4-chloro-1,2-diphenyl-1-butenyl]- | | Pharmaceutical Corp., |
| phenoxy)-N,N-dimethylethylamine citrate (1:1)) | | Eatontown, NJ |
| Tositumomab, I 131 Tositumomab | Bexxar | Corixa Corp., Seattle, |
| (recombinant murine immunotherapeutic | | WA |
| monoclonal IgG₂ₐ lambda anti-CD20 antibody (I | | |
| 131 is a radioimmunotherapeutic antibody)) | | |
| Trastuzumab | Herceptin | Genentech, Inc |
| (recombinant monoclonal IgG₁ kappa anti-HER2 | | |
| antibody) | | |
| Tretinoin, ATRA (all-trans retinoic acid) | Vesanoid | Roche |
| Uracil Mustard | Uracil Mustard | Roberts Labs |
| | Capsules | |
| Valrubicin, N-trifluoroacetyladriamycin-14-valerate | Valstar | Anthra --> Medeva |
| ((2S-cis)-2-[1,2,3,4,6,11-hexahydro-2,5,12- | | |
| trihydroxy-7 methoxy-6,ll-dioxo-[[42,3,6- | | |
| trideoxy-3-[(trifluoroacetyl)-amino-α-L-lyxo- | | |
| hexopyranosyl]oxyl]-2-naphthacenyl]-2-oxoethyl | | |
| pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′- | | |
| norvincaleukoblastine [R—(R*,R*)-2,3- | | |
| dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

Anticancer agents further include compounds which have been identified to have anticancer activity but are not currently approved by the U.S. Food and Drug Administration or other counterpart agencies or are undergoing evaluation for new uses. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG000011, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflornithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafarnib, luniliximab, mafosfamide, MB07133, MDX-010, MLN₂₇O₄, monoclonal antibody 3F8, monoclonal antibody J591, motexafin, MS-275, MVA-MUC1-IL2, nilutamide, nitrocamptothecin, nolatrexed dihydrochloride, nolvadex, NS-9, 06-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RADOO1, ranpirnase, rebeccamycin analogue, rhuAngiostatin protein, rhuMab 2C4, rosiglitazone, rubitecan, S-1, S-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifarnib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

Preferred conventional anticancer agents for use in administration with the present compounds include, but are not limited to, adriamycin, 5-fluorouracil, etoposide, camptothecin, actinomycin D, mitomycin C, cisplatin, docetaxel, gemcitabine, carboplatin, oxaliplatin, bortezomib, gefitinib, and bevacizumab. These agents can be prepared and used singularly, in combined therapeutic compositions, in kits, or in combination with immunotherapeutic agents, and the like.

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” ninth edition, Eds. Hardman et al., 1996.

The present invention provides methods for administering apogossypolone or pharmaceutically acceptable salts or prodrugs thereof with radiation therapy. The invention is not limited by the types, amounts, or delivery and administration systems used to deliver the therapeutic dose of radiation to an animal. For example, the animal may receive photon radiotherapy, particle beam radiation therapy, other types of radiotherapies, and combinations thereof. In some embodiments, the radiation is delivered to the animal using a linear accelerator. In still other embodiments, the radiation is delivered using a gamma knife.

The source of radiation can be external or internal to the animal. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by patients. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

The animal may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to a patient, so long as the dose of radiation is tolerated by the patient without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. The dose of radiation is preferably fractionated for maximal target cell exposure and reduced toxicity.

The total dose of radiation administered to an animal preferably is about 0.01 Gray (Gy) to about 100 Gy. More preferably, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), preferably 1-2 Gy (e.g., 1.5-2 Gy). The daily dose of radiation should be sufficient to induce destruction of the targeted cells. If stretched over a period, radiation preferably is not administered every day, thereby allowing the animal to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the animal's responsiveness and any potential side effects. Radiation therapy can be initiated at any time in the therapeutic period. Preferably, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the present invention.

Antimicrobial therapeutic agents may also be used as therapeutic agents in the present invention. Any agent that can kill, inhibit, or otherwise attenuate the function of microbial organisms may be used, as well as any agent contemplated to have such activities. Antimicrobial agents include, but are not limited to, natural and synthetic antibiotics, antibodies, inhibitory proteins (e.g., defensins), antisense nucleic acids, membrane disruptive agents and the like, used alone or in combination. Indeed, any type of antibiotic may be used including, but not limited to, antibacterial agents, antiviral agents, antifungal agents, and the like.

In some embodiments of the present invention, inhibitors of anti-apoptotic Bcl-2 family proteins, such as apogossypolone or salts or prodrugs thereof and one or more therapeutic agents or anticancer agents are administered to an animal under one or more of the following conditions: at different periodicities, at different durations, at different concentrations, by different administration routes, etc. In some embodiments, apogossypolone is administered prior to the therapeutic or anticancer agent, e.g., 0.5, 1, 2, 3, 4, 5, 10, 12, or 18 hours, 1, 2, 3, 4, 5, or 6 days, or 1, 2, 3, or 4 weeks prior to the administration of the therapeutic or anticancer agent. In some embodiments, apogossypolone is administered after the therapeutic or anticancer agent, e.g., 0.5, 1, 2, 3, 4, 5, 10, 12, or 18 hours, 1, 2, 3, 4, 5, or 6 days, or 1, 2, 3, or 4 weeks after the administration of the anticancer agent. In some embodiments, apogossypolone or salts or prodrugs thereof and the therapeutic or anticancer agent are administered concurrently but on different schedules, e.g., apogossypolone or salts or prodrugs thereof are administered daily while the therapeutic or anticancer agent is administered once a week, once every two weeks, once every three weeks, or once every four weeks. In other embodiments, apogossypolone or salts or prodrugs thereof are administered once a week while the therapeutic or anticancer agent is administered daily, once a week, once every two weeks, once every three weeks, or once every four weeks.

The compounds of the present invention may be linked to a carrier molecule to enhance the cellular uptake of the compounds. Examples of such carrier molecules include carrier peptides such as those described by Fulda et al., Nature Med. 8:808 (2002), Arnt et al., J. Biol. Chem. 277:44236 (2002), and Yang et al., Cancer Res. 63:831 (2003), fusogenic peptides (see, e.g., U.S. Pat. No. 5,965,404), and viruses and parts of viruses such as empty capsids and virus hemagglutinin (see, e.g., U.S. Pat. No. 5,547,932). Other carrier molecules include ligands for cell surface receptor such as asialoglycoprotein (which binds to the asialoglycoprotein receptor; see U.S. Pat. No. 5,166,320) and antibodies to cell surface receptors such as antibodies specific for T-cells, e.g., anti-CD4 antibodies (see U.S. Pat. No. 5,693,509).

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. Preferably, about 0.01 to about 10 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, and most preferably, from about 0.01 to about 5 mg/kg.

The unit oral dose may comprise from about 0.01 to about 50 mg, preferably about 0.1 to about 10 mg of the compound. The unit dose may be administered one or more times daily as one or more tablets or capsules each containing from about 0.1 to about 10, conveniently about 0.25 to 50 mg of the compound or its solvates.

In a topical formulation, the compound may be present at a concentration of about 0.01 to 100 mg per gram of carrier. In a preferred embodiment, the compound is present at a concentration of about 0.07-1.0 mg/ml, more preferably, about 0.1-0.5 mg/ml, most preferably, about 0.4 mg/ml.

In addition to administering apogossypolone or salts or prodrugs thereof or other inhibitors of anti-apoptotic Bcl-2 family proteins as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. Preferably, the preparations, particularly those preparations which can be administered orally or topically and which can be used for the preferred type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by injection, topically or orally, contain from about 0.01 to 99 percent, preferably from about 0.25 to 75 percent of active compound(s), together with the excipient.

The pharmaceutical compositions of the invention may be administered to any animal which may experience the beneficial effects of the compounds of the invention. Foremost among such animals are mammals, e.g., humans, although the invention is not intended to be so limited. Other animals include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal, or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Creams are preferably formulated from a mixture of mineral oil, self-emulsifying beeswax and water in which mixture the active ingredient, dissolved in a small amount of an oil such as almond oil, is admixed. A typical example of such a cream is one which includes about 40 parts water, about 20 parts beeswax, about 40 parts mineral oil and about 1 part almond oil.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight.

Lotions may be conveniently prepared by dissolving the active ingredient, in a suitable high molecular weight alcohol such as propylene glycol or polyethylene glycol.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### EXAMPLE 1

### Fluorescence Polarization Binding Assay

Fluorescence polarization (FP)-based binding assays for Bcl-2 and Bcl-xL were developed and optimized using recombinant human Bcl-2 and Bcl-xL proteins, a Bid BH3 peptide labeled with 6-carboxyfluorescein succinimidyl ester (FAM) and Bak BH3 peptide labeled with 6-(fluorescein-5(6)-carboxamido) hexanoic acid (Flu). The FP-based binding assays measure the ability of an inhibitor to displace either Bid-FAM or Bak-Flu peptide from Bcl-2 or Bcl-xL protein, respectively. The dose-dependent binding experiments were carried out with serial dilutions of the tested compounds in DMSO. For the Bcl-2 binding assay, a 5 μl sample of the inhibitor and preincubated recombinant His-fused soluble Bcl-2 protein (120 nM) with Bid-FAM peptide (10 nM) in the assay buffer (100 mM potassium phosphate, pH 7.5; 100 μg/ml bovine gamma globulin; 0.02% sodium azide, purchased from Invitrogen, Life Technologies), were added in Dynex 96-well, black, round-bottom plates (Fisher Scientific), to produce a final volume of 125 μl. A total of 15 different concentrations of the inhibitor were typically used to determine the IC₅₀ value from the plot using a nonlinear least-square analysis and curve fitting performed using GraphPad Prism® software. The polarization values were measured after 3-4 hr incubation, using an ULTRA READER (Tecan U.S. Inc., Research Triangle Park, N.C.). The Kᵢ value was calculated using a modified Cheng-Prusoff equation based upon the K_{d} value for the Bid-FAM peptide to Bcl-2, the measured IC₅₀ value, and the concentrations of the protein and Bid-FAM. For the Bcl-xL binding assay, the recombinant human Bcl-xL fused to a His-tag without the C-terminus hydrophobic tail and the Bak-Flu peptide were used. The Bcl-xL assay was performed in a similar manner as the Bcl-2 assay except 60 nM of Bcl-xL and 5 nM of Bak-Flu peptide were used in assay buffer (50 mM Tris-Bis, pH 7.4; 0.01% bovine gamma globulin). Unlabeled Bid and Bak peptides, (−)-gossypol and apogossypolone were used as the positive controls. An inactive analogue of gossypol was used as the negative control.

### EXAMPLE 2

### Design of Gossypol Analogs

(−)-Gossypol has been shown to bind to Bcl-2 and Bcl-xL at the BH3 binding groove and to have significant anticancer activity (U.S. Patent Application No. 2003/0008924). (−)-Gossypol contains two reactive aldehyde groups in its structure. These two reactive groups form Schiffs bases with lysine residues in proteins and have been attributed to the toxicity of gossypol in animals and humans. This toxicity, albeit mild, limits the maximum dose that can be given to patients. Extensive efforts were utilized to identify gossypol analogs that are both less toxic and bind more tightly to Bcl-2.

Two analogs, gossypolic acid (V) and gossypolonic acid (VI), were designed to maintain the interaction between the aldehyde group and an arginine residue in Bcl-2 and Bcl-xL (Arg-139 and Arg-141, respectively). These two compounds were determined to have Kᵢ values of 120 nM and 280 nM, respectively, i.e., slightly more potent than (−)-gossypol. However, the two acid groups in these compounds are negatively charged at physiological conditions (pH=7.4), and this may prevent them from entering cells. In assays for inhibition of cell growth in PC-3 cells, both compounds had IC₅₀ values greater than 10 μM.

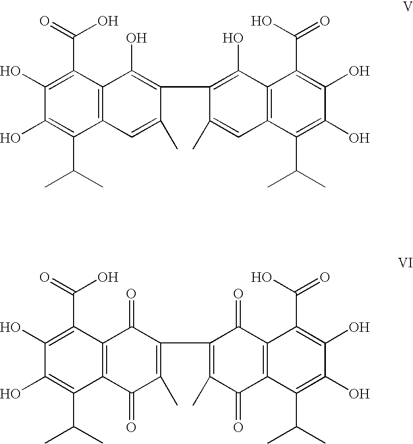

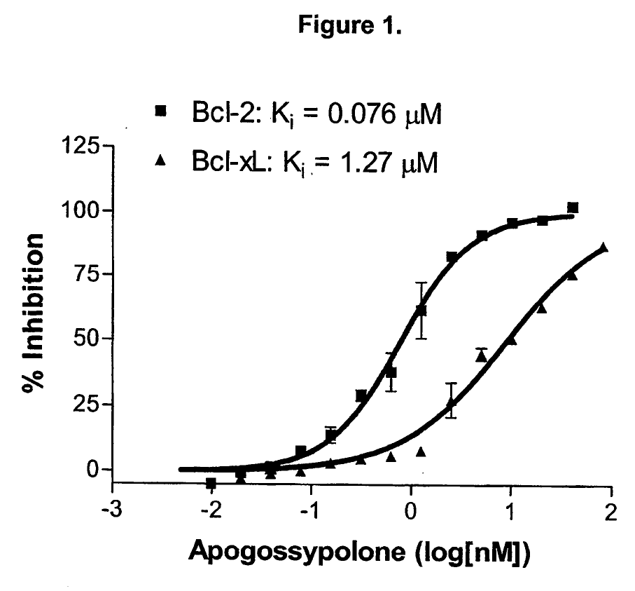

To overcome the lower cell-permeability of gossypolic acid and gossypolonic acid, two more compounds were designed and synthesized, apogossypol (VII) and apogossypolone (I), in which the two aldehyde groups of gossypol are completely removed. Apogossypol and apogossypolone were determined to have Kᵢ values of 200 nM and 76 nM, respectively. The binding curve for apogossypolone to Bcl-2 is shown in FIG. 1. The Kᵢ value for apogossypolone to Bcl-xL was determined to be 1.27 μM (FIG. 1). Hence, apogossypolone represents a potent small-molecule inhibitor.

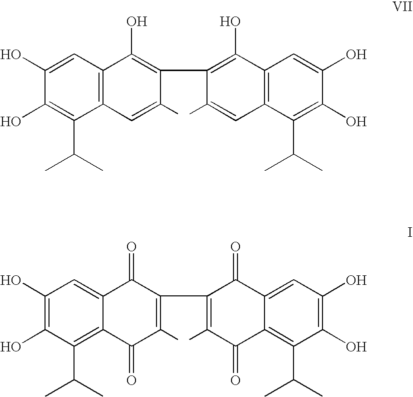

### EXAMPLE 3

### Cell Growth Inhibition Activity of Apogossypolone

A direct comparison of (−)-gossypol, apogossypol and apogossypolone for their activity in inhibition of cell growth in PC-3 and LnCap prostate cancer cell lines was carried out. In 3-5 independent experiments, apogossypol was as potent as (−)-gossypol, while apogossypolone (IC₅₀=2.2 μM) was consistently 3-4 times more potent than (−)-gossypol (IC₅₀=6.5 μM) in inhibition of cell growth in a 4-6 day MTT assay in the PC-3 cell line. Similar results were found with the LnCap cell line (apogossypolone IC₅₀=1.3 μM, (−)-gossypol IC₅₀=4.7 μM). However, apogossypol was very unstable and the sample rapidly decomposed within 1 week even stored at −20° C. and under nitrogen. In contrast, apogossypolone was very stable; no decomposition was detected when it was stored at room temperature for several weeks without the protection of nitrogen.

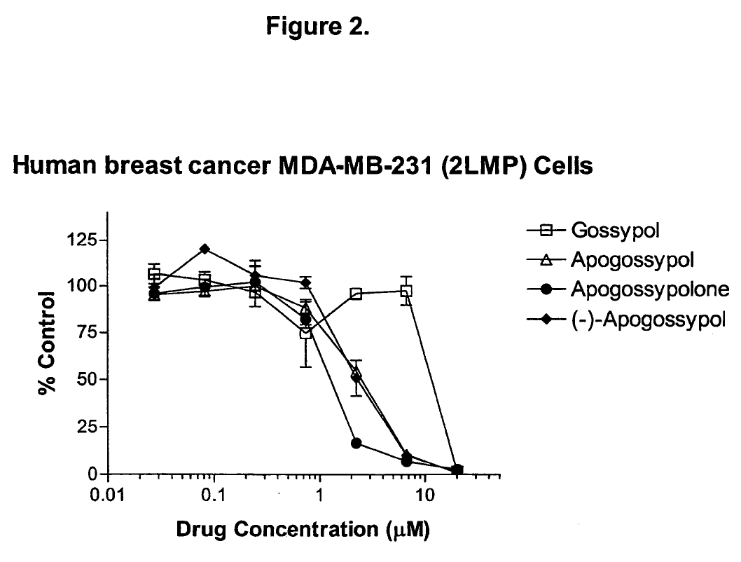

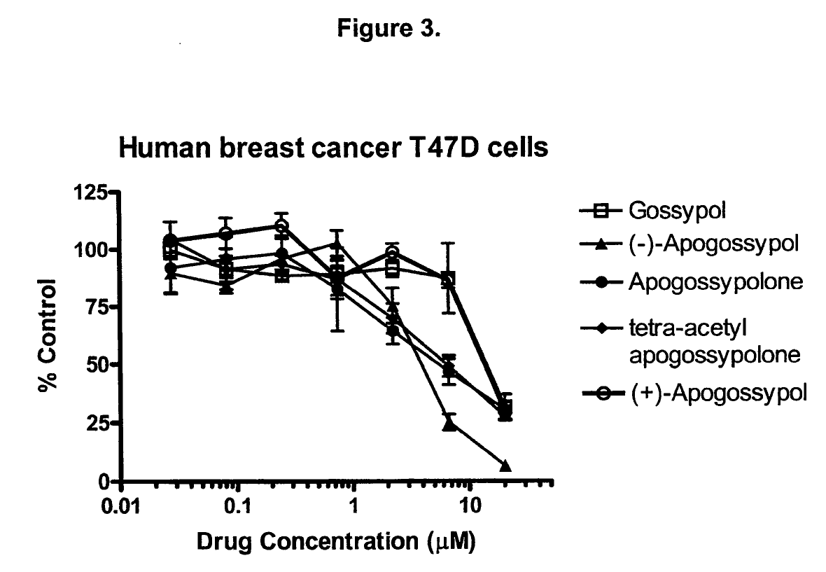

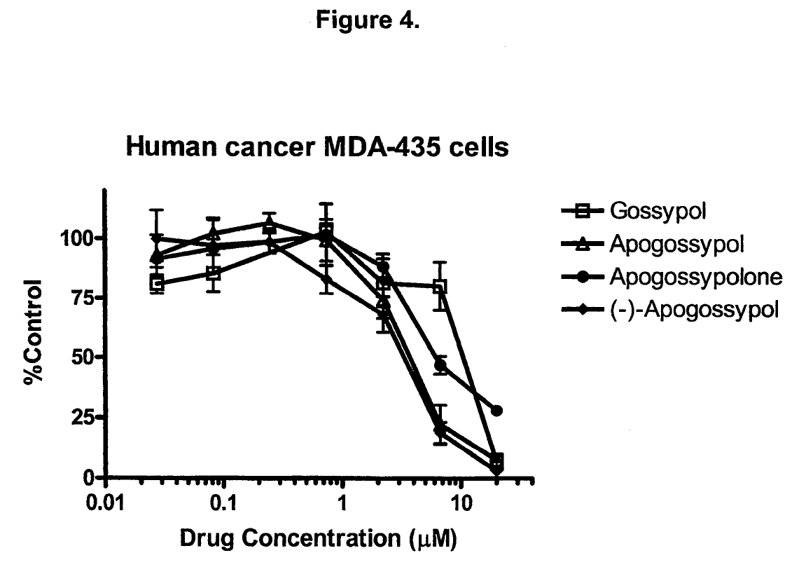

Additional cell growth inhibition studies were performed using the MDA-MB-231 (subclone 2LMP), T47D, and MDA-MB-435 breast cancer cell lines. Racemic gossypol, apogossypol, apogossypolone, (+)-apogossypol, (−)-apogossypol, and tetra-acetyl apogossypolone (Formula VIII) were tested as described above. A summary of the results is provided in Table 2. In the MDA-MB-231 cell line apogossypol was about 5-fold more potent than gossypol while apogossypolone was about 9-fold more potent than gossypol (FIG. 2). In the T47D cell line apogossypol was about 2.5-fold more potent than gossypol while apogossypolone was about 2-fold more potent than gossypol (FIG. 3). In the MDA-MB-435 cell line apogossypol was about 3-fold more potent than gossypol while apogossypolone was about 2-fold more potent than gossypol (FIG. 4).

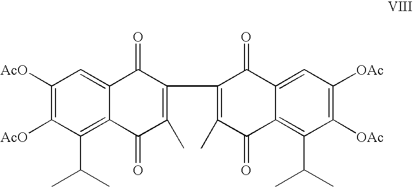

TABLE 2

| | IC₅₀ (μM) | | |
|---|---|---|---|
| Compound | MDA-MB-231 | MDA-MB-435 | T47D |
| (±)-Gossypol | 11.12 | 11.38 | 16.29 |
| Apogossypolone | 1.27 | 4.82 | 9.26 |
| Tetra acetyl | 1.32 | 6.29 | 6.33 |
| apogossypolone | | | |
| Apogossypol | 2.37 | 3.39 | 6.51 |
| (+)-Apogossypol | 8.81 | 7.28 | 14.00 |
| (−)-Apogossypol | 2.31 | 3.13 | 4.26 |

### EXAMPLE 4

### Toxicity of Apogossypolone

During the development of the present invention, it was contemplated that the major toxicity of gossypol in animals and humans is associated with its two reactive aldehyde groups and that removal of these aldehydes should significantly reduce the toxicity. To confirm this prediction, the maximal tolerated dose (MTD) of apogossypolone and (−)-gossypol was evaluated in mice using two different routes of administrations (oral and intravenous). For oral administration (oral gavage 5 days/week), the MTD of apogossypolone was above 240 mg/kg while the MTD for (−)-gossypol was about 50 mg/kg. For intravenous administration (every other day and 3 days/week), the MTD of apogossypolone was about 80 mg/kg while the MTD for (−)-gossypol was about 10 mg/kg. In both routes of administration, the MTD of apogossypolone was 8-times better than (−)-gossypol. Hence, apogossypolone is well tolerated in mice and is much less toxic than (−)-gossypol.

### EXAMPLE 5

### Antitumor Activity of Apogossypolone

An in vivo study of apogossypolone was carried out using a PC-3 xenograft model in nude mice to evaluate the antitumor activity of apogossypolone alone or in combination with X-ray irradiation. Nude mice with established PC-3 xenografts (150 mm³ in size) were treated with (1) apogossypolone 200 mg/kg p.o. q.d. 5×4 weeks; (2) fractionated X-ray irradiation to the tumor only, with the animal body shielded, at a dose of 2 Gy q.d. 5×3 weeks, total 30 Gy; and (3) a combination of apogossypolone and radiation.

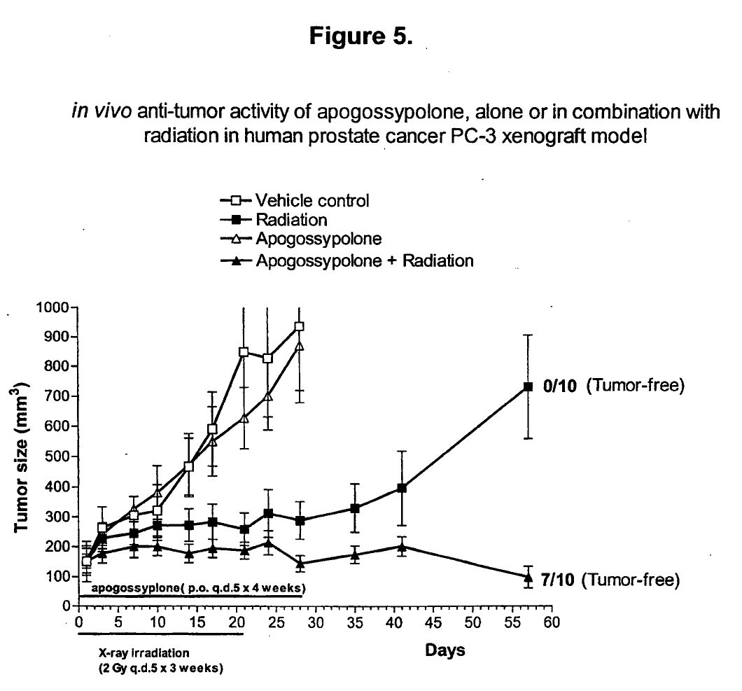

As shown in FIG. 5, apogossypolone alone had a limited effect on the tumors. However, apogossypolone significantly enhanced the radiation-mediated tumor inhibition (P<0.0001, two-way ANOVA, n=10). More significantly, on Day 57, the apogossypolone plus radiation treatment achieved complete tumor regression in seven out of ten tumors whereas either treatment alone resulted in zero out of ten complete regressions. The data indicated that apogossypolone is a potent radiosensitizer and finds use as a therapeutic agent for treating advanced, hormone refractory prostate cancer and other diseases.

### EXAMPLE 6

### Synthesis of Apogossypolone

(+)-Apogossypolone was prepared from gossypol acetic acid as shown in Scheme I above.

### (+)-5,5′-Diisopropyl-3,3′-dimethyl-[2,2′]binaphthalenyl-1,6,7,1′,6′,7′-hexaol (II)

Gossypol acetic acid (6.0 g, 10.4 mmol) was heated in 40% aqueous sodium hydroxide (40 ml) at 85° C. under nitrogen atmosphere for 2 h. The reaction mixture was poured onto ice containing concentrated sulfuric acid. The resultant precipitate was extracted with ether, and the combined extracts were washed with water, dried, and concentrated in vacuo to yield crude II, which was used directly for the next step without further purification.

### Acetic acid 1,7,1′,6′,7′-pentaacetoxy-5,5′-diisopropyl-3,3′-dimethyl-[2,2′]binaphthalenyl-6-yl Ester (III)

Acetic anhydride (7.8 ml, 83.2 mmol) was added to a solution of crude II in dichloromethane (100 ml), followed by N,N′-diisopropylethylamine (14.5 ml, 83.2 mmol). The reaction mixture was stirred at room temperature for 12 h. The reaction was quenched by addition of water. Chloroform was added, the layers were separated, and the aqueous phase was extracted twice with chloroform. The combined extracts were washed with brine, dried, and concentrated in vacuo. Flash silica gel column chromatography (2% acetone/chloroform) afforded 6.1 g of III as a pale yellow solid, yield 82% for two steps.

### Acetic acid 6,6′,7′-triacetoxy-5,5′-diisopropyl-3,3′-dimethyl-1,4,1′,4′-tetraoxo-1,4,1′,4′-tetrahydro-[2,2′]binaphthalenyl-7-yl Ester (IV)

Periodic acid (20 g, 87.7 mmol) was added to a solution of III (2.0 g, 2.8 mmol) in dioxane (30 ml) and the reaction mixture was stirred at 95° C. for 15 min. Crushed ice was added to quench the reaction. Ethyl acetate was added, the layers were separated, and the aqueous phase was extracted twice with ethyl acetate. The combined extracts were washed with brine, dried, and concentrated in vacuo. Flash silica gel column chromatography (2% acetone/chloroform) afforded 0.65 g of IV as a bright yellow solid, yield 35%.

### 6,7,6′,7′-Tetrahydroxy-5,5′-diisopropyl-3,3′-dimethyl-[2,2′]binaphthalenyl-1,4,1′,4′-tetraone,apogossypolone (I)

A 10% solution of potassium carbonate (10 ml) was added to a solution of IV (0.6 g, 0.91 mmol) in dioxane (15 ml) and the reaction mixture was stirred at 70° C. for 5 h. After cooling, 4 M HCl was added to the solution and the pH was adjusted to 5. Ethyl acetate was added, the layers were separated, and the aqueous phase was extracted twice with ethyl acetate. The combined extracts were washed with brine, dried, and concentrated in vacuo. Recrystallization from ethyl acetate/hexane afforded 0.44 g of apogossypolone (I) as a brown yellow solid, yield 98%.

### EXAMPLE 7

### Synthesis of Gossypolonic Acid

(+)-Gossypolonic acid was prepared from gossypol acetic acid by reported methods (Rogers et al., J. Am. Chem. Soc. 60:2170 (1938)) as shown in Scheme III above. Racemic gossypol was converted to hexamethyl ether using dimethyl sulfate in the presence of methanol and potassium hydroxide. The hexamethyl ether was oxidized to gossypolonic acid tetramethyl ether by dilute nitric acid. Methyl ester was obtained by dimethyl sulfate in refluxing acetone in the presence of potassium carbonate. All six methyl groups were removed by boron tribromide in dichloromethane at −20° C., followed by workup with dilute aqueous acetic acid to give gossypolonic acid as a yellow solid.

¹HNMR (300 MHz, CDCl₃) δ 7.62 (s, 2H), 6.04 (s, 2H), 4.26 (m, J=6.9 Hz, 2H), 2.00 (s, 6H), 1.39 (m, 12H).

### EXAMPLE 8

Fluorescence Polarization Binding Assay for the Mcl-1 Protein

### Human Mcl-1 Protein Expression and Purification

Human Mcl-1 cDNA was purchased from Origene. The fragment encoding amino acids 171-327 was cloned into the pHis-TEV vector (a modified pET vector) through BamHIH and EcoRI sites, using the oligos: 5′-CGGGATCCGAGGACGAGTTGTACCGGCAG-3′ (SEQ ID NO:1) and 5′-GGAATTCCTAGCCACCTTCTAGGTCCTCTAC-3′ (SEQ ID NO:2). Mcl-1 171-327 aa protein with a N-terminal 8×His tag was produced in E. coli BL21(DE3) cells. Cells were grown at 37° C. in 2xYT containing antibiotics to an OD₆₀₀ density of 0.6. Protein expression was induced by 0.4 mM IPTG at 37° C. for 4 hours. Cells were lysed in 50 mM Tris pH 8.0 buffer containing 500 mM NaCl, 0.1% bME and 40 μl of Leupectin/Aprotin. Mcl-1 171-327 aa protein was purified from the soluble fraction using Ni-NTA resin (QIAGEN), following the manufacturer's instruction. The protein was further purified on a Source Q15 column (resin and column are from Amersham Biosciences) in 25 mM Tris pH 8.0 buffer, with NaCl gradient. Purified protein was aliquoted and stored at −80° C. in the presence of 25% glycerol.

### Fluorescence Polarization Binding Assay

A sensitive and quantitative in vitro fluorescence polarization-based (FP) binding assay was optimized and used to determine the in vitro binding affinity of apogossypolone against Mcl-1 protein, a Bcl-2 family protein member.

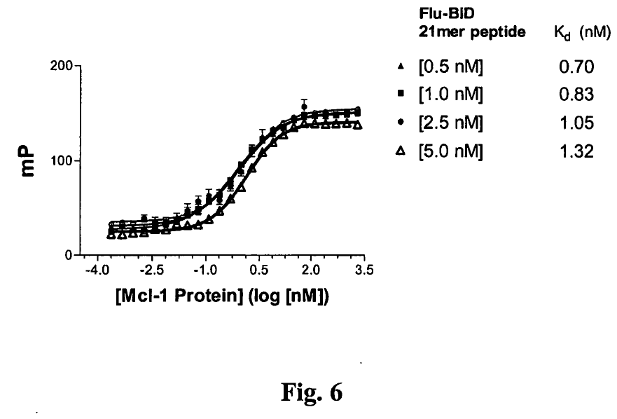

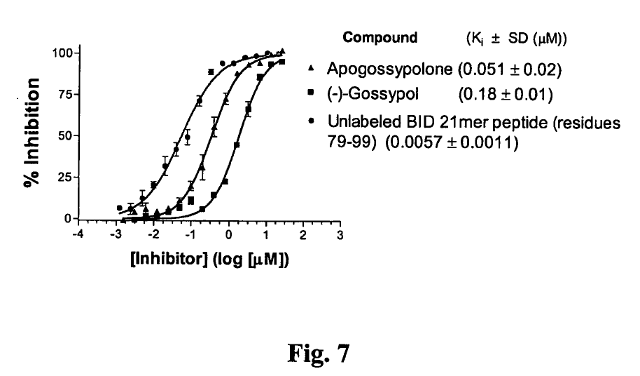

In Vitro Mcl-1 binding assay. An FP-based method was established and optimized to test the binding affinity of apogossypolone against Mcl-1 protein. For this assay a 21-residue Bid BH3 peptide (QEDIIRNIARHLAQVGDSMDR (SEQ ID NO:3)) labeled at the N-terminus with 6-carboxyfluorescein succinimidyl ester (FAM) was used as the fluorescence tag (Flu-Bid-21). The dissociation constant (K_{d}) for the complex Flu-Bid-21 probe and Mcl-1 protein was determined using a constant concentration of probe, 1 nM, and titrating with the Mcl-1 protein at increasing concentrations significantly above the expected K_{d} of the protein-probe pair. FIG. 6 illustrates nonlinear least-squares fits to a single-site binding model for a saturation experiment in which the Mcl-1 protein concentration varied from 0 to 2 μM at constant probe concentration. The fluorescent probe, Flu-Bid-21, shows high binding affinity for Mcl-1 protein with K_{d}=0.83 nM and dynamic range of ΔmP=122 mP (ΔmP=mP of bound peptide−mP of free peptide). The possibility of a change in K_{d} values when the concentration of the probe is reduced was explored. In principle, when the probe concentration is above the true K_{d} value, a higher probe concentration will result in a higher apparent K_{d} value (Kenakin, Pharmacological Analysis of Drug-Receptor Interaction, Lippincott-Raven, Philadelphia (1997)). Under four concentrations of probe Flu-Bid-21 (5, 2.5, 1 and 0.5 nM), apparent K_{d} values of 1.32, 1.05, 0.83 and 0.70 nM, respectively, were obtained for the fluorescent probe (FIG. 6). These results thus indicated that the apparent K_{d} value for the probe obtained under each of the four concentrations approaches the true K_{d} value. Assay specificity was confirmed by competitive displacement of the labeled Flu-Bid 21mer binding to Mcl-1 by an unlabeled Bid 21mer peptide with Kᵢ=5.7±1.1 nM, which is in good agreement with determined K_{d} (FIG. 7).

The dose-dependent competitive binding experiments were carried out with serial dilutions of the tested compounds in DMSO. A 5 μl sample of the tested samples and preincubated Mcl-1 protein (5 nM) and Flu-Bid-21mer peptide (1 nM) in the assay buffer (100 mM potassium phosphate, pH 7.5; 100 μg/ml bovine gamma globulin; 0.02% sodium azide, purchased from Invitrogen™ Life Technology), were added in Dynex 96-well, black, round-bottom plates (Fisher Scientific) to produce a final volume of 125 μl. For each assay, the controls included the Mcl-1 protein and Flu-Bid-21mer peptide (equivalent to 0% inhibition) and only Flu-Bid-21mer peptide (equivalent to 100% inhibition). The polarization values were measured after 3 hrs of incubation using an ULTRA READER (Tecan U.S. Inc., Research Triangle Park, N.C.). The IC₅₀ values, i.e. the inhibitor concentration at which 50% of bound peptide is displaced, were determined from a plot using nonlinear least-squares analysis. Curve fitting was performed using GRAPHPAD PRISM software (GraphPad Software, Inc., San Diego, Calif.). The Kᵢ values were calculated using our developed equation for FP assay, including the influence of the excess of protein in the competitive binding assay:

Kᵢ=[I]₅₀/([L]₅₀/K_{d}+[P]₀/K_{d}+1)

where [I]₅₀ denotes the concentration of the free inhibitor at 50% inhibition, [L]₅₀ is the concentration of the free labeled probe at 50% inhibition, [P]₀ is the concentration of the free protein at 0% inhibition, and K_{d} is the dissociation constant of the protein-ligand complex. This equation was derived from the basic principles of a competitive binding assay and also it was derived the solutions of all parameters required in this new equation for accurately computing of the Ki values of inhibitors (Nikolovska-Coleska et al., Anal. Biochem. 332: 261 (2004)).

Using the FP based assay the binding affinity of apogossypolone was determined. The Kᵢ value for apogossypolone to Mcl-1 was determined to be 0.051±0.02 μM and the binding curve is shown in FIG. 7. Apogossypolone, as a gossypol analog, shows 3.5 fold better binding affinity compared with (−)-gossypol, Kᵢ=0.18±0.01 (FIG. 7).

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

1-32. (canceled)

33. A method of inducing apoptosis in a cell comprising contacting said cell with a compound selected from the group consisting of apogossypolone, (−)-apogossypolone, and (+)-apogossypolone, or a pharmaceutically acceptable salt or prodrug thereof.

34. A method of rendering a cell sensitive to an inducer of apoptosis comprising contacting said cell with a compound selected from the group consisting of apogossypolone, (−)-apogossypolone, and (+)-apogossypolone, or a pharmaceutically acceptable salt or prodrug thereof.

35. The method of claim 34, further comprising contacting said cell with an inducer of apoptosis.

36. The method of claim 35, wherein said inducer of apoptosis is a chemotherapeutic agent.

37. The method of claim 35, wherein said inducer of apoptosis is radiation.

38. A method of treating, ameliorating, or preventing a disorder responsive to the induction of apoptosis in an animal, comprising administering to said animal a therapeutically effective amount of a compound selected from the group consisting of apogossypolone, (−)-apogossypolone, and (+)-apogossypolone, or a pharmaceutically acceptable salt or prodrug thereof.

39. The method of claim 38, further comprising administering to said animal an inducer of apoptosis.

40. The method of claim 39, wherein said inducer of apoptosis is a chemotherapeutic agent.

41. The method of claim 39, wherein said inducer of apoptosis is radiation.

42. The method of claim 39, wherein said disorder responsive to the induction of apoptosis is a hyperproliferative disease.

43. The method of claim 42, wherein said hyperproliferative disease is cancer.

44. The method of claim 39, wherein said compound is administered prior to said inducer of apoptosis.

45. The method of claim 39, wherein said compound is administered concurrently with said inducer of apoptosis.

46. The method of claim 39, wherein said compound is administered after said inducer of apoptosis.

47. A method of treating, ameliorating, or preventing a hyperproliferative disease in an animal, comprising administering to said animal a therapeutically effective amount of a compound selected from the group consisting of apogossypolone, (−)-apogossypolone, and (+)-apogossypolone, or a pharmaceutically acceptable salt or prodrug thereof.

48. The method of claim 47, wherein said hyperproliferative disease is cancer.

49. The method of claim 47, further comprising administering to said animal an inducer of apoptosis.

50. The method of claim 49, wherein said inducer of apoptosis is a chemotherapeutic agent.

51. The method of claim 49, wherein said inducer of apoptosis is radiation.


SEQUENCE LISTING (3 sequences)

SEQ ID NO: 1 - DNA, 29 bp, Artificial Sequence
  Synthetic
cgggatccgaggacgagttgtaccggcag

SEQ ID NO: 2 - DNA, 31 bp, Artificial Sequence
  Synthetic
ggaattcctagccaccttctaggtcctctac

SEQ ID NO: 3 - protein, 21 aa, Artificial Sequence
  Synthetic
QEDIIRNIARHLAQVGDSMDR